(19) United States
(12) Patent Application Publication
(10) Pub. No.: US 2024/0215819 A1
(43) Pub. Date: Jul. 4, 2024

(54) OPTICAL MEASUREMENT APPARATUS AND METHOD OF SCANNING A FOCUS

(71) Applicant: Occuity Limited, London (GB)

(72) Inventors: James REYNOLDS, Gloucestershire (GB); Robin TAYLOR, Berkshire (GB)

(73) Assignee: Occuity Limited, London (GB)

(21) Appl. No.: 18/563,770

(22) PCT Filed: May 16, 2022
(86) PCT No.: PCT/EP2022/063183

§ 371 Date: Nov. 22, 2023

(30) Foreign Application Priority Data
May 26, 2021 (GB) GB2107470.3

(51) Int. Cl. A61B 3/10; A61B 3/14

(52) U.S. Cl. CPC A61B 3/1005; A61B 3/14

(57) ABSTRACT

An optical measurement apparatus (100) comprises a point source (102) of electromagnetic radiation and an optical path extending from the point source of electromagnetic radiation to a window (122). An optical input arrangement (108) is also provided and comprises a static input lens. An optical output arrangement (114) of the apparatus comprises a static output lens. The apparatus further comprises an intermediate lens (112) and a scanning mechanism (110) configured to carry the intermediate lens (112). A position measurement system is operably coupled to the scanning mechanism (110) and configured to measure a position of the intermediate lens (112). The optical input arrangement (108), the intermediate lens (112) and the optical output arrangement (114) are disposed in the optical path, and the intermediate lens (112) and the scanning mechanism (110) are disposed between the optical input arrangement (108) and the optical output arrangement (114). The scanning mechanism (110) is configured to scan positionally linearly the intermediate lens (112).

The present invention relates to an optical measurement apparatus of the type that, for example, measures thickness of a region to be measured located at a substantially static target location. The present invention also relates to a method of scanning a focus, the method being of the type that, for example, scans a focus of an optical measurement apparatus for measurement of a thickness a region to be measured located at a substantially static target location.

In the field of optical metrology, it is desirable to measure a thickness of a region of a material under investigation, for example biological tissue. For some applications, for example and not limited to optical pachymetry, it is necessary to scan an eye at a frequency higher than the speed of involuntary movements of the eye so that the eye is effectively static over the duration of a single measurement of the eye, thereby eliminating errors attributable to movement of the eye.

A typical measurement system to perform optical measurements, for example pachymetry, comprises a confocal optical system having, inter alia, a light source, a collimating lens and an output lens group disposed within a housing, the housing having a measurement window therein located opposite the output lens group. The lens group comprises an objective lens and the light source, the collimating lens and the output lens group are all arranged in an optical train of the optical system. An object to be measured, for example an eye, is offered up to the measurement window for measuring the thickness of the cornea of the eye. Typically, the objective lens is reciprocated in order to scan the focus of the optical system. However, the objective lens has to be appropriately dimensioned in order to support a relatively large working range and output numerical aperture of the optical system. As a result of these design considerations, it is necessary to specify a sufficiently large diameter for the objective lens and so the objective lens is inevitably bulky and thus relatively heavy. The mass of the objective lens therefore presents difficulties in achieving sufficiently high scan speeds, for example achieving a sufficiently high scan speed without introducing an intolerable degree of vibration into the optical system that will introduce measurement inaccuracies into the measurement system.

Another known technique comprises scanning a light source of the optical system mentioned above. Whilst lighter than a typical objective lens, where the light source is an end of an optical fibre, the mass of the fibre optic light source is still significant and can only be scanned at limited speeds. Furthermore, the optical fibre nevertheless has to be connected physically to a static object, for example a laser diode. This physical connection causes additional loading of a translatable carriage employed to scan the end of the optical fibre. Furthermore, applying repetitive strain to the connection to the static object can shorten the working life of the light source.

In confocal microscopy, it is known to employ so-called relay lenses, which are static, for scanning in a plane at a fixed depth. Japanese laid-open publication no. JP 2000111801A describes a supplemental confocal unit for a microscope and discloses the use of a relay lens. Similarly, Japanese laid-open publication nos. JP 2013167654, JP2008249965 and JP2007286310 each disclose the use of a relay lens in relation to microscopy.

U.S. Pat. No. 10,302,925 B2 employs an electrically tuneable lens in a variable focal element to change a focal depth of a microscope. Japanese laid-open publication no. JP 2020021014A similarly describes a static liquid lens to provide a variable focal lens. However, in all of the examples set forth above, either heavy optical elements are translated for adjustment purposes and so the challenges of high speed scanning of weighty optical elements is not encountered, or translation to adjust the focal depth is avoided altogether by employing less conventional lens types as relay optics in order to adjust, but not scan, the focal depth of the optical system.

According to a first aspect of the present invention, there is provided an optical measurement apparatus comprising: a point source of electromagnetic radiation; an optical input arrangement comprising a static input lens; an optical output arrangement comprising a static output lens; an optical path extending from the point source of electromagnetic radiation to an output surface of the optical output arrangement; an intermediate lens; a scanning mechanism configured to carry the intermediate lens; and a position measurement system operably coupled to the scanning mechanism and configured to measure a position of the intermediate lens; wherein the optical input arrangement, the intermediate lens and the optical output arrangement are disposed in the optical path; the intermediate lens and the scanning mechanism are disposed between the optical input arrangement and the optical output arrangement; and the scanning mechanism is configured to scan positionally linearly the intermediate lens.

The intermediate lens may be configured to provide, when in use, a virtual source of electromagnetic radiation. The scanning of the intermediate lens by the scanning mechanism may be configured to scan the virtual source of electromagnetic radiation relative to the optical output arrangement.

The scanning mechanism may be an electromechanical scanning mechanism.

The intermediate lens may be a relay lens.

The optical output arrangement may comprise a plurality of optical elements; the plurality of optical elements may be static. The optical output arrangement may be a static optical output arrangement.

The optical path may be folded, for example substantially right-angled or bent in shape.

The apparatus may comprise a bidirectional optical transceiver. The bidirectional optical transceiver may be a packaged component. The bidirectional optical transceiver may comprise a source of electromagnetic radiation and an optical detector. The bidirectional optical transceiver may comprise a beamsplitter and an optical fibre output. Alternatively, instead of the bidirectional optical transceiver, a source of electromagnetic radiation and an optical detector may be provided, the source of electromagnetic radiation and the optical detector may be respectively operably coupled to an optical circulator or optical splitter; an optical fibre output may also be operably coupled to the optical circulator or optical splitter to provide the point source of electromagnetic radiation.

The scanning mechanism may be configured to translate reciprocatably the intermediate lens.

The scanning mechanism may be configured to translate reciprocatably the intermediate lens a plurality of times within a scan window. The plurality of times may be greater than two.

The optical input arrangement may be an optical collimating arrangement.

The static input lens may be a collimating lens.

The optical input arrangement may comprise an optical element. The optical element may be a silica rod.

The scanning mechanism may be configured to translate over a measurement epoch at a speed greater than a speed of movement of an eye.

The scanning mechanism may be configured to reciprocate at a frequency greater than 20 Hz. The frequency may be greater than 25 Hz, for example greater than 50 Hz, 100 Hz or 200 Hz.

The apparatus may further comprise: a source of electromagnetic radiation operable coupled to a first end of an optical fibre; wherein the point source of electromagnetic radiation may be a second end of the optical fibre.

The intermediate lens may be formed from a plastics material. The intermediate lens may be an aspheric lens. The intermediate lens may be formed from an optical-grade plastics material. The intermediate lens may be diffraction limited. The intermediate lens may have a diameter less than substantially 10 mm, for example substantially 6 mm.

The apparatus may further comprise: a processing resource configured to support a calibration module; and a position encoder device operably coupled to the scanning mechanism to provide a measured position of the intermediate lens; wherein the optical output arrangement may be configured to generate an output focal point; and the calibration module may be configured to relate the measured position of the intermediate lens to a position of the output focal point.

The calibration module may comprise a modelling unit configured to relate the measured position of the intermediate lens to the position of the output focal point. The modelling unit may be configured to generate an output focal position data point in response to a measured position data point using a mathematical formula.

The apparatus may further comprise: a lookup table comprising position data in respect of the scanning mechanism and output focal position data; wherein the lookup table may be configured to record a correspondence between the position data and output focal position data; and the calibration module may be configured to access the lookup table and extract an output focal position data entry by reference to a position data entry of the lookup table.

The correspondence between the position data and the output focal position data of the lookup table may be configured to comprise correction of nonlinearities in measurement of a position of the intermediate lens by the position measurement system as well as compensation for a nonlinear relationship between the position of intermediate lens and the position of the output point.

The scanning mechanism may be configured to scan the intermediate lens over a range of up to about 10 mm. The scanning mechanism may be configured to scan the intermediate lens over a range of at least 1 mm, 5 mm or 10 mm, for example about 8 mm.

A first magnitude of a first focal length of the intermediate lens may be less than a second magnitude of a second focal length of the optical input arrangement.

A numerical aperture of the intermediate lens may be greater than a numerical aperture of the optical input arrangement.

The optical output arrangement may comprise a plurality of optical elements; the plurality of optical elements may be configured to provide a symmetric arrangement of the plurality of optical elements.

The optical input arrangement, the intermediate lens and the optical output arrangement may be configured to target laterally a substantially central location in space; the optical output arrangement may have an output numerical aperture between a minimum output numerical aperture necessary to scan within a predetermined area comprising the substantially central location and a maximum output numerical aperture to maintain substantially consistent measurement results over the predetermined area.

The output numerical aperture may be between about 0.16 and about 0.4, for example between about 0.17 and about 0.3, such as about 0.2.

The apparatus may be configured to guide light therein using optical elements, for example optical fibre. The apparatus may comprise one or more optical fibres organised with one or more optical component as a network and configured to operably couple the point source to the optical input arrangement.

The apparatus may comprise an optical detector unit operably coupled to the optical input arrangement. The network of optical fibres may be configured to operably couple the optical detector unit to the optical input arrangement.

The optical measurement apparatus may be a confocal optical measurement apparatus.

According to a second aspect of the present invention, there is provided an ophthalmic measurement apparatus comprising the optical measurement apparatus as set forth above in relation to the first aspect of the present invention.

The ophthalmic measurement apparatus may be a pachymeter.

According to a third aspect of the present invention, there is provided a handheld optical measurement apparatus comprising the optical measurement apparatus as set forth above in relation to the first aspect of the present invention.

According to a fourth aspect of the present invention, there is provided a method of scanning a focus of an optical measurement apparatus, the method comprising: generating and emitting source electromagnetic radiation; directing the electromagnetic radiation so as to provide an internal virtual point source using an intermediate lens; scanning the internal virtual point source; monitoring a position of the intermediate lens; and statically focussing the divergent electromagnetic radiation to an output focus.

The electromagnetic radiation may be focussed to provide the internal virtual point source. The focussed electromagnetic radiation may diverge following focussing to provide divergent electromagnetic radiation.

The method may further comprise: translating a measured position of the intermediate lens to a position of the output focus.

It is thus possible to provide an apparatus and method that provide rapid scanning of an output focus in the z-direction of an optical system whilst avoiding scanning of a large diameter optical element of the optical system at high speed. As a consequence of being able to scan the intermediate lens at a higher frequency than conventional heavy output optical elements, undesirable vibrations that contaminate measurements being made are thereby obviated or at least mitigated. The scanning of the intermediate lens also permits the point source of electromagnetic radiation to remain static. The intermediate lens forms, when in use, a virtual source of electromagnetic radiation and so the numerical aperture of the virtual source of electromagnetic radiation is selectable owing to the power of the intermediate lens being selectable. The design flexibility of the optical system is therefore also improved. The use of calibration to compensate for a nonlinear relationship between a measured position of the intermediate lens and the output focal point is, contrary to many application, not an undesirable necessary processing overhead, because the calibration can provide a twofold benefit of accounting for said nonlinearity, but also providing an ability to accommodate different mapping ratios between the position of the intermediate lens and the output focal point, thereby facilitating adjustment of an output scanning range of the optical system and resolution of the output focal point. Resolution of the optical system can therefore be selected so as to offset accuracy of measurement against cost of parts.

At least one embodiment of the invention will now be described, by way of example only, with reference to the accompanying drawings, in which:

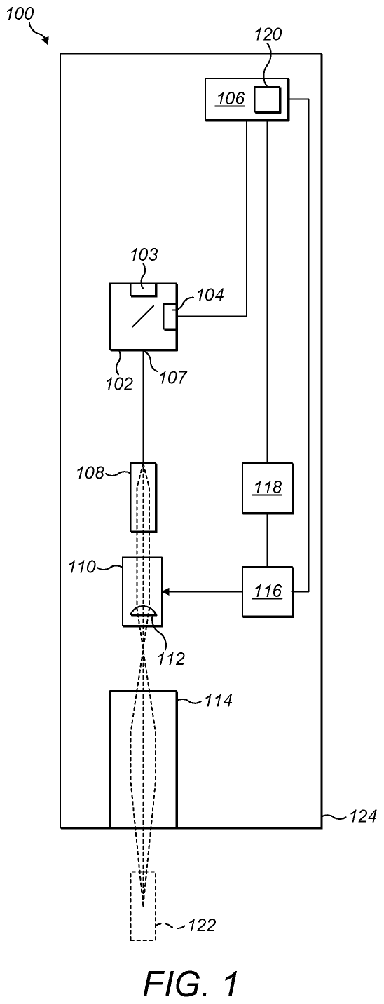

FIG. 1 is a schematic diagram of an optical measurement apparatus constituting an embodiment of the invention;

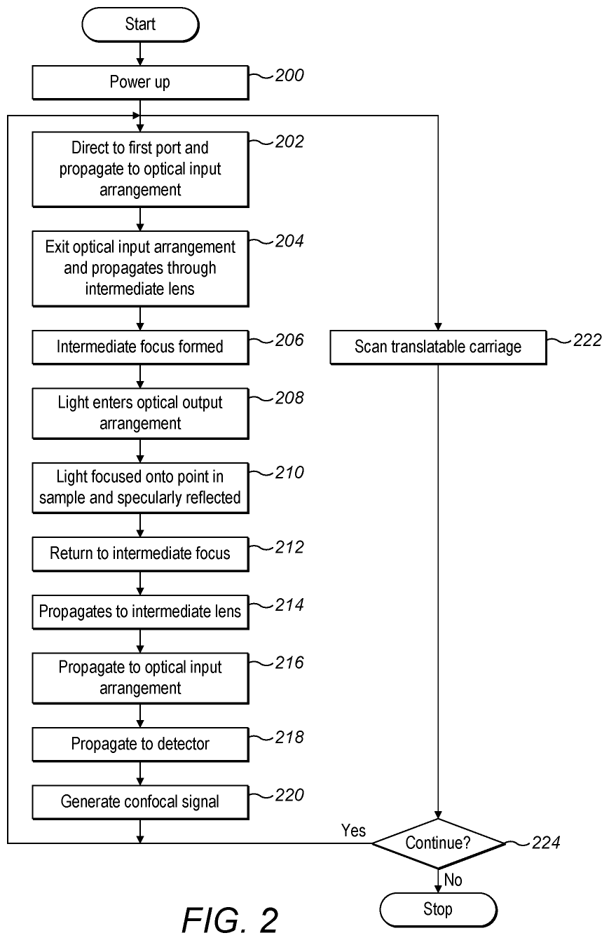

FIG. 2 is a flow diagram of a method of measurement using the apparatus of FIG. 1; and

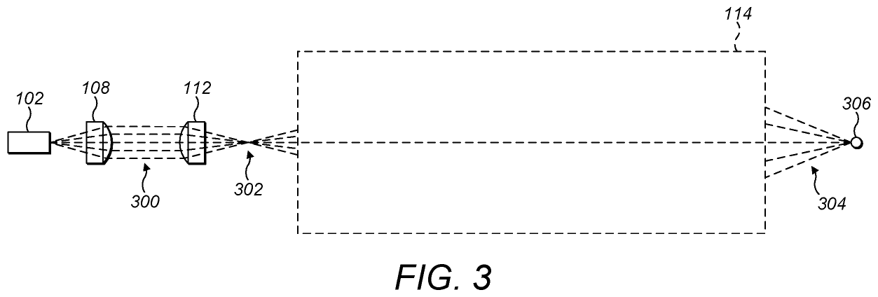

FIG. 3 is a ray diagram of the apparatus of FIG. 1.

Throughout the following description identical reference numerals will be used to identify like parts.

Referring to FIG. 1, an optical measurement apparatus 100 comprises a bidirectional optical transceiver 102 comprising a source of electromagnetic radiation, for example a Laser Diode (LD) 103, an optical detector 104, for example a photodiode, the optical detector 104 being operably coupled to a confocal detector unit 106. The bidirectional optical transceiver 102 can be any suitable arrangement of optical and optoelectronic devices employing a light source and a photodiode. An example of a suitable bidirectional optical transceiver is described in UK patent no. 2 508 368. The optical detector 104 in combination with the confocal detector unit 106 are configured, in this example, to generate signal peaks in respect of electromagnetic radiation backscattered from boundaries of regions with different refractive index in a sample 122 under test. As the exact implementation of the confocal detector unit 106 is not core to an understanding of the operation of the embodiments described herein, the confocal detector unit 106 will not be described in any further detail.

An input/output port of the bidirectional optical transceiver 102 is operably coupled to a first port of an optical input arrangement 108 by optical fibre at a first end thereof. In this example, the optical input arrangement 108 is a fibre collimator that couples the input/output port 107 of the bidirectional optical transceiver 102 via a second end of the optical fibre. However, it should be appreciated that any other suitable implementation of the optical input arrangement 108 can be employed, for example a separate collimating lens or the input optical arrangement can comprise a collimating lens. In these examples, the optical input arrangement 108 is an optical collimating arrangement and comprises a static input lens, which in some examples is the collimating lens. The use of the optical fibre to couple input/output port 107 to the optical input arrangement 108 serves to provide an actual point source of the electromagnetic radiation. In this example, the bidirectional optical transceiver 102 permits electromagnetic radiation emitted by the source of electromagnetic radiation 103 to pass through to the input/output port 107 thereof and electromagnetic radiation received at the input/output port 107 is directed to the optical detector 104. In other implementations, an optical launch assembly, as described in European patent no. EP-B-2 391 262, can be provided. The optical input arrangement 108 can comprise the optical launch assembly, which can be operably coupled to the second end of the optical fibre described herein to reduce reflections back into the optical fibre. The optical launch assembly can comprise a ferrule, split sleeve, silica rod and an index matching medium for example a gel or adhesive. The silica rod and the ferrule can optionally be angle polished.

A second port of the optical input arrangement 108 is disposed opposite a first side of a linearly translatable carriage 110 carrying, in this example, an optical element, for example an intermediate lens 112. In some examples, the intermediate lens 112 constitutes a relay lens. In this example, the intermediate lens 112 is formed from a plastics material, such as an optical-grade plastics material. The intermediate lens 112 is, in this example, an aspheric lens and can be diffraction limited. In this example, the intermediate lens 112 has a diameter of about 6 mm, but the diameter of the intermediate lens 112 can be less than about 10 mm, thereby ensuring that the intermediate lens 112 is lightweight and suitable for undergoing rapid movement at the frequencies set forth later herein.

The intermediate lens 112 lens is mounted on the translatable carriage 110 and is also disposed opposite a first port of an optical output arrangement 114. Although one translatable lens is described in the examples herein, the skilled person should appreciate that at least one optical element can be employed. The linearly translatable carriage 110 constitutes an electromechanical scanning mechanism, for example of the kind described in copending UK patent application no. 2019190.4. However, the skilled person should appreciate that the manner of driving the translatable carriage 110 need not be electromechanical and any other suitable technique can be employed, for example employing a pneumatic drive mechanism. The optical output arrangement 114 can be any suitable arrangement of optical elements. The optical elements include a static output or projector lens, for example an objective lens.

A processing resource, for example a translation controller 116 such as a microcontroller, is operably coupled to the translatable carriage 110. The translatable carriage 110 carries an encoder scale and a linear encoder is disposed opposite the encoder scale and operably coupled to the translation controller 116. The combination of the linear encoder and the encoder scale is, for example, of the type described in UK patent no. GB 2 467 340, and serves to provide position feedback, when in use, with respect to the translatable carriage 110. The processing resource also supports a measurement unit 118 operably coupled to the translation controller 116 as well as the confocal detector unit 106. The linear encoder, the encoder scale and the measurement unit 118 constitute a position measurement system. In this example, the processing resource also supports a calibration module 120 to relate the output of the linear encoder to a position in space of a focus of the optical output arrangement 114 over a region to be scanned in which the sample 122, at least partially, resides.

The calibration module 120 can comprise a modelling unit (not shown) to relate the measured position of the intermediate lens 112 to the position of the output focal point. The modelling unit can be configured to generate an output focal position data point in response to a measured position data point using a mathematical formula. In this regard, a curve fitting technique can be employed by the calibration module 120 to fit, for example, an analytical function to the error correction values calculated. One suitable technique is a polynomial best fit technique. The fitted curve can then be referenced by the calibration module 120 to determine a corresponding focal distance of a spot generated by the optical output arrangement 114. However, in this example, the processing resource supports a lookup table comprising position data in respect of the translatable carriage 110 and corresponding output focal position data corresponding to position along the optical axis extending through the optical input arrangement 108, the intermediate lens 112, the optical output arrangement 114, and the region to be scanned. The lookup table records a correspondence between the position data and the output focal position data that can be provided to the confocal detector unit 106 in order to determine, for example, an actual distance between peaks detected by the confocal detector unit 106.

The sample 122, for example the biological tissue, which can be in vivo or in vitro, is disposed opposite a second port of the optical output arrangement 114. An outermost facing surface of the second port of the optical output arrangement 114 is substantially flush with a housing 124 to permit electromagnetic radiation to propagate from the second port of the optical output arrangement 114 directly to the sample 122 before being backscattered, for example specularly reflected, by the sample 122 back to the second port of the output optical arrangement. The distance of the sample 122 from the second port of the optical output arrangement 114 is substantially static with respect to a single measurement time window.

The housing 124 contains, in this example, the bidirectional optical transceiver 102, the laser diode 103, the optical detector 104, the confocal detector unit 106, the optical input arrangement 108, the translatable carriage 110, the intermediate lens 112, the optical output arrangement 114, the translation controller 116, the measurement unit 118, the calibration module 120, the encoder scale and the linear encoder.

As will be apparent to the person skilled in the art, the bidirectional optical transceiver 102, the laser diode 103, the optical detector 104, the confocal detector unit 106, the optical input arrangement 108, the intermediate lens 112, and the optical output arrangement 114 constitute, in this example, a confocal measurement system. However, other configurations of optical and/or optoelectronic elements can be employed for different measurement applications requiring an intermediate scanning focus. In this example, an optical path is defined from the actual point source of electromagnetic radiation to the outermost facing surface of the second port of the optical output arrangement 114 via the optical input arrangement 108, the intermediate lens 112, and the optical output arrangement 114. The optical path is redirected, for example by a beamsplitter of the bidirectional optical transceiver 102. In this regard, the optical path is not straight and can be folded, for example the optical path can comprise a right-angle in respect of one or both of the emission and reception directions.

Referring to FIG. 3, the optical output arrangement 114 comprises a plurality of optical elements (not shown). In some examples, the plurality of optical elements can be configured as a symmetric arrangement of optical elements. In this example, the plurality of optical elements is static. In this example, the optical output arrangement 114 comprises a projection lens (not shown), for example an objective lens. The projection lens is static in this example. The optical output arrangement 114 serves to create a secondary focus, which constitutes a confocal point 306, within the region to be scanned, to be described later herein. The intermediate lens 112 has a first focal length associated therewith and the optical output arrangement 114 has a second focal length associated therewith, the magnitude of the first focal length being less than the magnitude of the second focal length. Also, in this example, the intermediate lens 112 has a first numerical aperture associated therewith and the optical input arrangement 108 has a second numerical aperture associated therewith, the first numerical aperture being greater than the second numerical aperture.

In the context of a human eye, as the anterior chamber and the posterior chamber of the eye have different radii of curvature, thickness of the cornea changes with lateral position relative to the optical axis. As such, for accurate measurement, it is necessary to measure within a relatively central region of the cornea without deviating too far from a central location on the cornea. As such, the optical input arrangement 108, the intermediate lens 112 and the optical output arrangement 114 are configured to scan longitudinally the region to be scanned, but are also configured to receive optical signals laterally over a substantially central but limited location in space through which the optical axis of the extends, for example a central location of the cornea of the eye, constituting the sample 122. In this regard, in a general sense, the cornea can be any curved surface over which measurements are to be made. In order to limit the lateral deviation, an output numerical aperture of the optical output arrangement 114 is limited to a maximum output numerical aperture value. Additionally, and as measured above, light reflected from the cornea result in peaks that are detected by the confocal detector unit 106. If the output numerical aperture is set too low, the peaks generated from neighbouring surfaces causing reflections begin to coalesce, making detection of distinct peaks problematic or even impossible. Therefore, in order to maintain a minimum resolution in this regard to enable the peaks to be distinctly detected, the output numerical aperture of the optical output arrangement 114 is set to a minimum output numerical value. The output numerical aperture is, in this example, between about 0.16 and about 0.4, for example between about 0.17 and about 0.3, such as about 0.2.

In operation (FIGS. 2 and 3), the laser diode 103, the optical detector 104 and the confocal detector unit 106 are powered up (Step 200) and electromagnetic radiation, for example monochromatic electromagnetic radiation (hereafter referred to as “output light”) is emitted by the laser diode 103. The output light emitted by the laser diode 103 passes through the bidirectional optical transceiver 102 to the input/output port 107 thereof and is therefore directed (Step 202) to the optical input arrangement 108 where the output light is collimated. The collimated output light 300 is incident upon the intermediate lens 112 (Step 204), which focusses (Step 206) the collimated output light to an intermediate focus 302 prior to entering the output optical arrangement 114. The intermediate focus 302 provided by the intermediate lens 112 constitutes a virtual source of electromagnetic radiation. The output light then diverges before entering the optical output arrangement 114 and propagating through the optical output arrangement 114. The output light propagating through the optical output arrangement 114 is conditioned by the optical elements contained therein before then being focussed 304 (Step 210) onto a point 306, for example a near-diffraction limited spot, within the region to be scanned via the outermost facing surface of the second port of the optical output arrangement 114, the region being scanned comprising, at least in part, the sample 122.

The focus 306 lies within a region to be measured as defined by the design parameters of the optical measurement apparatus 100, for example the optical input arrangement 108, the intermediate lens 112, and the optical output arrangement 114. The sample 122 is, in this example substantially static, in particular in the z-direction (depth), during a measurement epoch, for example the optical measurement apparatus 100 is not provided with a mechanism to move the sample 122 relative to the housing 124 and hence the optical output arrangement 114.

The conditioned output light 304 focussed onto or into the sample 122 is specularly reflected (Step 210) and some of the light (hereinafter referred to as the “returning light”) is focussed (Step 212) to the intermediate focal point 302 by the optical output arrangement 114, whereupon the returning light diverges and propagates (Step 214) to the intermediate lens 112 and is collimated thereby. The collimated reflected light then propagates (Step 216) to and through the input optical arrangement 108 and is focussed thereby. The returning light then propagates to the bidirectional optical transceiver 102, whereupon the returning light is directed (Step 218) by the bidirectional optical transceiver 102 to the optical detector 104 coupled to the confocal detector unit 106.

The confocal detector unit 106 receiving the electrical signals generated by the optical detector 104 generates (Step 220) a confocal detection signal. The confocal detection signal is analysed in accordance with any suitable confocal measurement technique, for example to detect peaks in the confocal detection signal. As the exact nature of the measurement being performed by the optical measurement apparatus 100 is not central to an understanding of the salient aspects of the examples set forth herein, for the sake of clarity and conciseness of description, the operation of the confocal detector unit 106 will not be described in further detail.

During irradiation of the sample 122 and generation of the confocal detection signal, the translatable carriage 110 is controlled by the translation controller 116 to scan (Step 222) the intermediate lens 112 towards and away from the optical output arrangement 114 over a predetermined range of travel and at a predetermined frequency, thereby varying the confocal detection signal. In this regard, the translatable carriage 110 is configured to translate the intermediate lens 112 reciprocatably. The scanning of the intermediate lens 112 by the translatable carriage 110 is positionally linear. The scanning of the intermediate lens 112 serves to scan the intermediate focus 302 and thus the virtual source of electromagnetic radiation, for example relative to the optical output arrangement. In this regard, it should be appreciated that in this example a measurement epoch comprises a single translation or sweep in one direction of the intermediate lens 112 while the intermediate lens 112 is oscillating. In this regard, as the translatable carriage 110 and the intermediate lens 112 are oscillating, a measurement epoch is, in this example, substantially one half of the oscillation period. However, a temporal scan window, during which multiple measurements are made can comprise more than one measurement epoch, and in this regard the measurement unit 118 can be configured to make a plurality of measurements during the temporal scan window. In other examples, a measurement epoch can comprise a cycle of the translatable carriage 110 towards and away from the optical output arrangement 114. The translatable carriage 110 is configured to reciprocate at a frequency greater than a speed of movement of the sample 122, which can move in some examples at a considerable speed and so the speed of the measurement epoch has to be sufficiently fast for any movement of the sample 122, for example an eye, so be sufficiently small not to impact the accuracy of the measurement being made. The frequency of reciprocation of the translatable carriage 110 can be greater than about 20 Hz, for example greater than about 25 Hz. Indeed, the speed of reciprocation of the translatable carriage can be greater than about 50 Hz, or greater than about 100 Hz or even greater than about 200 Hz.

It should be appreciated that the optical measurement apparatus 100 measures longitudinally. In this regard, the intermediate lens 112 constitutes a longitudinal imaging component. During translation of the translatable carriage 110, the translation controller 116 receives position information in respect of the translatable carriage 110 from the position encoder (not shown) for subsequent use in connection with the confocal measurement technique mentioned above. In this regard, the calibration unit 120 accesses the lookup table and obtains a spatial position for the focal point 306 corresponding to the position data generated by the position encoder. The position data and output focal position data contained in the lookup table comprise compensations for nonlinearities in the measurement of the position of the translatable carriage 110 by the position encoder and/or nonlinearities in the relationship between measured positions of the translatable carriage 110 and the actual position of the focal point 306. Although the need for calibration can be seen as disadvantageous, the use of the calibration unit 120 in the examples herein provides support to incorporate scaling factors other than 1:1 into the relationships between the measured positions and the actual positions of the focal point 306. For example, with appropriate choice of fixed optics in the optical output arrangement 114, a scan range of the confocal point 306 can be increased or decreased compared to that of the intermediate lens 112, and the resolution of the position of the confocal point 306 can thus be decreased or increased for a fixed resolution of position of the intermediate lens 112. In this example, the scan range of the translatable carriage 110 and hence the intermediate lens 112 is about 8 mm, but in other examples the scan range can be up to at least 1 mm, 5 mm or 10 mm.

The above measurement technique continues until a sufficient number of measurements has been made (Step 224) by the optical measurement apparatus 100 as defined by a measurement protocol of the optical measurement apparatus 100.

The skilled person should appreciate that the above-described implementations are merely examples of the various implementations that are conceivable within the scope of the appended claims. Indeed, the above examples relate to a confocal measurement apparatus. However, it should be appreciated that the principle of reciprocatably translating an intermediate lens multiple times so as to generate a correspondingly reciprocating intermediate focus to serve as a virtual source of electromagnetic radiation can be employed in other measurement apparatus, for example ophthalmic measurement apparatus. The optical measurement apparatus 100 can be employed in a pachymeter in some embodiments. Furthermore, the optical measurement apparatus 100 is suitable for disposing in a compact housing, for example in a handheld optical measurement apparatus.

Although reference has been made in the above examples to an optical “input” arrangement and an optical “output” arrangement, it should be appreciated that the terms “input” and “output” are simply being employed for the sake of convenience using the direction of emission of electromagnetic radiation from the source of electromagnetic radiation 103 as a frame of reference. Indeed, the skilled person will also appreciate that the optical path is bidirectional owing to the specularly reflected light propagating back from the sample 122 to the confocal detector unit 106 and so the optical input arrangement 108 and the optical output arrangement 114 can simply be considered a first optical element arrangement and a second optical element arrangement, respectively, and separated by the intermediate lens 112.

Although collimation is employed in the above examples, the skilled person will appreciate that in other examples, collimation does not need to take place.

In the above examples, the bidirectional optical transceiver 102 is employed to direct the output light to the optical input arrangement and the returning light to the optical detector 104. However, in other examples, an optical circulator or a fibre splitter can be employed and coupled to the source of electromagnetic radiation 103 and the optical detector 104.

The above examples guide light between one or more of the optical elements, i.e. the implementation comprises one or more waveguides for optical interconnection purposes arranged with the optical element(s)/unit(s) of the apparatus 100, where appropriate, as a network. For example, as a minimum, an optical fibre interconnects the bidirectional optical transceiver 102 and the optical input arrangement 108. However, in other examples such as where an optical circulator or optical fibre splitter is employed, more fibre interconnections can be required between the optical circulator or optical fibre splitter, the source of electromagnetic radiation 103, the optical detector 104 and the optical input arrangement 108. It should also be appreciated that in the examples set forth herein, guiding materials can optionally be employed for electromagnetic radiation propagating from the optical fibre coupled to the bidirectional transceiver 102 and/or collimator to the intermediate lens 112 or from the intermediate lens 112 to the output optical arrangement 114. The skilled person will appreciate that a so-called “free space” implementation is possible where the bidirectional optical transceiver 102, which in the examples set forth herein is a packaged and/or self-contained component comprising the source of electromagnetic radiation 103, is replaced by an independent source of electromagnetic radiation, an independent optical detector and a beamsplitter that are not packaged in a self-contained manner and free-space is present between these components, i.e. they are not interconnected by a guiding material, such as optical fibre, for example air or any other gaseous medium is present between these components.

It should be appreciated that references herein to “light”, other than where expressly stated otherwise, are intended as references relating to the optical range of the electromagnetic spectrum, for example, between about 350 nm and about 2000 nm, such as between about 550 nm and about 1400 nm or between about 600 nm and about 1000 nm.

1. An optical measurement apparatus comprising:
a point source of electromagnetic radiation;
an optical input arrangement comprising a static input lens;
an optical output arrangement comprising a static output lens;
an optical path extending from the point source of electromagnetic radiation to an output surface of the optical output arrangement;
an intermediate lens;
a scanning mechanism configured to carry the intermediate lens; and
a position measurement system operably coupled to the scanning mechanism and configured to measure a position of the intermediate lens; wherein
the optical input arrangement, the intermediate lens and the optical output arrangement are disposed in the optical path;
the intermediate lens and the scanning mechanism are disposed between the optical input arrangement and the optical output arrangement; and
the scanning mechanism is configured to scan positionally linearly the intermediate lens.

2. The apparatus according to claim 1, wherein the scanning mechanism is configured to translate reciprocatably the intermediate lens.

3. The apparatus according to claim 1, wherein the optical input arrangement is an optical collimating arrangement.

4. The apparatus according to claim 1, wherein the scanning mechanism is configured to reciprocate at a frequency greater than 20 Hz.

5. The apparatus according to claim 1, further comprising:
a source of electromagnetic radiation operable coupled to a first end of an optical fibre; wherein
the point source of electromagnetic radiation is a second end of the optical fibre.

6. The apparatus according to claim 1, wherein the intermediate lens is formed from a plastics material.

7. The apparatus according to claim 1, further comprising:
a processing resource configured to support a calibration module; and
a position encoder device operably coupled to the scanning mechanism to provide a measured position of the intermediate lens; wherein
the optical output arrangement is configured to generate an output focal point; and
the calibration module is configured to relate the measured position of the intermediate lens to a position of the output focal point.

8. The apparatus according to claim 7, further comprising:
a lookup table comprising position data in respect of the scanning mechanism and output focal position data; wherein
the lookup table is configured to record a correspondence between the position data and output focal position data; and
the calibration module is configured to access the lookup table and extract an output focal position data entry by reference to a position data entry of the lookup table.

9. The apparatus according to claim 1, wherein the scanning mechanism being configured to scan the intermediate lens over a range of up to about 10 mm.

10. The apparatus according to claim 1, wherein a first magnitude of a first focal length of the intermediate lens is less than a second magnitude of a second focal length of the optical input arrangement.

11. The apparatus according to claim 1, wherein the optical output arrangement comprises a plurality of optical elements, the plurality of optical elements being configured to provide a symmetric arrangement of the plurality of optical elements.

12. The apparatus according to claim 1, wherein the optical input arrangement, the intermediate lens and the optical output arrangement are configured to target laterally a substantially central location in space, the optical output arrangement having an output numerical aperture between a minimum output numerical aperture necessary to scan within a predetermined area comprising the substantially central location and a maximum output numerical aperture to maintain substantially consistent measurement results over the predetermined area.

13. An ophthalmic measurement apparatus comprising an optical measurement apparatus, the optical measurement apparatus comprising:
a point source of electromagnetic radiation;
an optical input arrangement comprising a static input lens;
an optical output arrangement comprising a static output lens;
an optical path extending from the point source of electromagnetic radiation to an output surface of the optical output arrangement;
an intermediate lens;
a scanning mechanism configured to carry the intermediate lens; and
a position measurement system operably coupled to the scanning mechanism and configured to measure a position of the intermediate lens; wherein
the optical input arrangement, the intermediate lens and the optical output arrangement are disposed in the optical path;
the intermediate lens and the scanning mechanism are disposed between the optical input arrangement and the optical output arrangement; and
the scanning mechanism is configured to scan positionally linearly the intermediate lens.

14. A handheld optical measurement apparatus comprising the optical measurement apparatus according to claim 1.

15. A method of scanning a focus of an optical measurement apparatus, the method comprising:
generating and emitting source electromagnetic radiation;
directing the electromagnetic radiation so as to provide an internal virtual point source using an intermediate lens;
scanning the internal virtual point source;
monitoring a position of the intermediate lens; and
statically focussing the divergent electromagnetic radiation to an output focus.
